(19) United States
(12) Patent Application Publication
(10) Pub. No.: US 2023/0310438 A1
(43) Pub. Date: Oct. 5, 2023

(54) TLR7/8 AGONISTS TO ENHANCE IMMUNE RESPONSES IN OPIOID USING INDIVIDUALS

(71) Applicants: Children's Medical Center Corporation, Boston, MA (US); The University of Montana, Missoula, MT (US); Regents of the University of Minnesota, Minneapolis, MN (US)

(72) Inventors: David J. Dowling, Brighton, MA (US); Ofer Levy, Cambridge, MA (US); Dheeraj Soni, Brighton, MA (US); Sharon Levy, Cambridge, MA (US); Jay Evans, Corvallis, MT (US); Marco Pravetoni, Minneapolis, MN (US); David Burkhart, Missoula, MT (US); Shannon Miller, Missoula, MT (US)

(73) Assignees: Children's Medical Center Corporation, Boston, MA (US); the University of Montana, Missoula, MT (US); Regents of the University of Minnesota, Minneapolis, MN (US)

(21) Appl. No.: 17/769,037

(22) PCT Filed: Oct. 16, 2020
(86) PCT No.: PCT/US2020/056009

§ 371 Date: Apr. 14, 2022

Related U.S. Application Data
(60) Provisional application No. 62/916,735, filed on Oct. 17, 2019.

(51) Int. Cl. A61K 31/522; A61K 31/4745; A61K 31/7028; A61K 31/4468; A61K 39/00; A61P 25/36

(52) U.S. Cl. CPC A61K 31/522; A61K 31/4745; A61K 31/7028; A61K 31/4468; A61K 2039/55511; A61P 25/36; A61K 2039/6037; A61K 2039/6081; A61K 39/0013

(57) ABSTRACT

Provided herein are TLR7/8 agonists to enhance immune responses or for use as adjuvants in fentanyl vaccines in opioid-using individuals.

### RELATED APPLICATION

This Application claims the benefit under 35 U.S.C. § 119(e) of U.S. Provisional Application Ser. No. 62/916,735 entitled “TLR7/8 AGONISTS TO ENHANCE IMMUNE RESPONSES IN OPIOID USING INDIVIDUALS,” filed on Oct. 17, 2019, the entire contents of which are incorporated herein by reference.

### GOVERNMENT SUPPORT

This invention was made with government support under contract number HHSN272201800047C, HHSN272201800048C, and UG3-DA048386, awarded by the National Institutes of Health. The government has certain rights in this invention.

### BACKGROUND

The opioid epidemic has been declared a public health emergency with ˜130 Americans dying every day from an overdose. Fentanyl (FEN) contamination of heroin and other illicit drugs has accelerated the death rate. Adolescents and young adults with opioid use disorder (OUD) are at the epicenter of this crisis and have the highest rates of overdose deaths. Vaccines that block FEN have been developed but only demonstrated modest effects.

### SUMMARY

Provided herein are methods of enhancing immune response in a subject having an opioid use disorder (OUD), the method comprising administering to the subject an effective amount of a toll-like receptor 7 and 8 (TLR7/8) agonist and methods of treating an opioid use disorder (OUD) in a subject in need thereof, the method comprising administering to the subject an effective amount of a vaccine comprising an opioid antigen and a toll-like receptor 7 and 8 (TLR7/8) agonist. In some embodiments, the opioid antigen is fentanyl (FEN). In some embodiments, the opioid antigen is a fentanyl based hapten.

Some aspects of the present disclosure provide methods of treating an opioid use disorder (OUD) in a subject in need thereof, the method comprising administering to the subject an effective amount of a composition comprising an opioid antigen and a toll-like receptor 7 and 8 (TLR7/8) agonist.

In some embodiments, the opioid antigen is a fentanyl (FEN) antigen. In some embodiments, the FEN antigen is a fentanyl-based hapten. In some embodiments, the FEN antigen is a FEN conjugated to a carrier protein, optionally wherein the carrier protein is tetanus toxoid (TT), diphtheria toxoid (CRM), keyhole limpet hemocyanin (KLH).

In some embodiments, the TLR7/8 agonist is an imidazoquinoline compound. In some embodiments, the imidazoquioline compound is of Formula II:

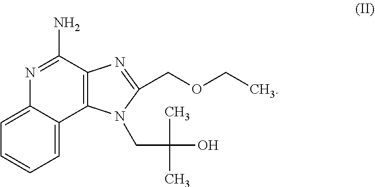 [SMILES: CCOCc1nc2c(N)nc3ccccc3c2n1CC(C)(C)O]

In some embodiments, the TLR7/8 agonist is an oxoadenine compound. In some embodiments, the oxoadenine compound is of Formula IV:

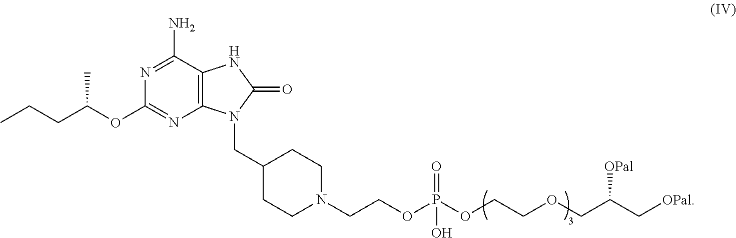

In some embodiments, the TLR7/8 agonist is lipidated. In some embodiments, the TLR7/8 agonist is incorporated into a liposome.

In some embodiments, the method further comprises administering to the subject an effective amount of a TLR4 agonist. In some embodiments, the TLR4 agonist is of Formula V:

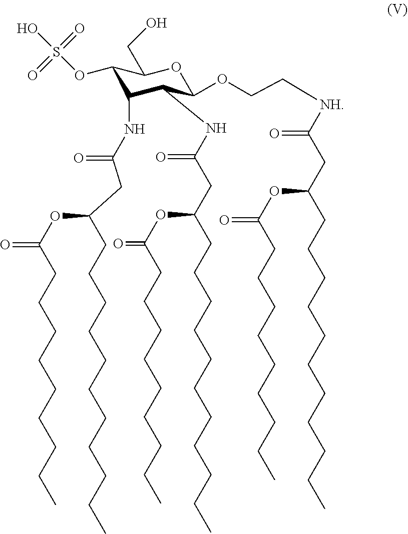

In some embodiments, the composition further comprises alum.

In some embodiments, the subject is a human adolescent. In some embodiments, the subject is a human adult. In some embodiments, the subject is a human that is between ages 18-30.

In some embodiments, the OUD is opioid addition. In some embodiments, the OUD is opioid tolerance. In some embodiments, the OUD is opioid overdose.

In some embodiments, the opioid is heroin, 6-acetylmorphine, morphine, oxycodone, hydrocodone, fentanyl, or analogs thereof.

In some embodiments, the administration is intravenous, intramuscular, intradermal, intranasal, topical, or oral. In some embodiments, the subject is administered a second agent for treating the OUD.

Further provided herein are compositions comprising an opioid antigen and a toll-like receptor 7 and 8 (TLR7/8) agonist. In some embodiments, the opioid antigen is fentanyl (FEN) or FEN based hapten. In some embodiments, the composition further comprises a TLR4 agonist. In some embodiments, the composition further comprises alum. In some embodiments, the composition is a vaccine.

Also provided herein are toll-like receptor 7 and 8 (TLR7/8) agonists for use as an adjuvant in an opioid vaccine, e.g., for enhancing an immune response in a subject having an opioid use disorder (OUD).

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various FIGS. is represented by a like numeral. For purposes of clarity, not every component may be labeled in every drawing. The patent or patent application file contains at least one drawing executed in color. Copies of this patent or patent application publication with color drawing(s) will be provided by the Office upon request and payment of the necessary fee.

In the drawings:

FIGS. 1A to 1C show the whole blood sample sparing methodology to study human immune phenotyping at the systems level, including transcriptomic, proteomic, metabolomic and single cell immunophenotyping from human blood sample volumes as low as <0.5 ml. This is an extension of a previously established work-flow (Lee et al., (2019) Nat Comm 10, 1092), FIG. 1A shows that after procuring of human blood (˜200 mL) from a given study participant (control or OUD), 500 μl of blood is immediately transferred to pre-stimulation-labelled Cryovial with PAXgene buffer. In addition, plasma is extracted from whole blood and aliquoted in different tubes (labelled for proteomics, ELISA, metabolomics and multiplex). FIG. 1B shows that out of 200 ml of blood collected, 150 ml is used for peripheral blood mononuclear cells (PBMCs) isolation. After isolation, PBMCs and platelet-poor plasma are cryopreserved for future experiments. FIG. 1C shows that a 24-well cell culture plate is used for setting up different stimulations in a whole-blood assay. 2 ml of blood is added per well with stimuli (either agonists, vaccines, or combination of vaccines and agonists). After 6 hrs of incubation, RNA and plasma samples are harvested (same as in FIG. 1A).

FIGS. 2A to 2I show that there are distinct immune responses to TLR4A in participants with OUD whole blood is overcome by the TLR7/8 agonist R848. Whole blood assay was setup for blood collected from adult donors with or without a history of Opioid use, Opioid User Disorder (OUD) and Control Cohort (CC), respectively. Blood was transferred to a 24-well plate, 2 ml/well mixed either with 0.2 ml DPBS alone (UnStim) or 0.2 ml of DPBS with agonists (Stim). Stimulation were DPBS alone (FIG. 2A), Engerix (1:10 v/v dilution) (FIG. 2B), Fentanyl-TT (1:5000 v/v dilution; ˜20 ng/ml) (FIG. 2C), Fentanyl-TT (same as in FIG. 2C) ad-mixture with Alum phosphate (10 μg/ml) (FIG. 2D), Synthetic-MPLA (100 ng/ml) (FIG. 2E), Synthetic-MPLA (100 ng/ml) ad-mixture with Fentanyl-TT (FIG. 2F), R848 (5 μM) (FIG. 2G), R848 (10 μM) ad-mixture with Fentanyl-TT (FIG. 2H). After 6 hours of incubation at 37° C., plasma was collected. All samples were run simultaneously on a FlexMAP 3D machine in a 96-well format for 14-plex cytokine readout. Graphs represent fold change compared to cytokine level in healthy donors' unstimulated sample. The values represent average cytokine level from n=3 for each group. FIG. 2I shows IL-12p40 as an example for impaired responses of youth with OUD, with ** indicating significance of 0.01, by non-parametric Mann-Whitney t-test.

FIGS. 3A to 3C show fentanyl-specific IgG antibody titers in a murine model in response to no immunization (naive), immunization with fentanyl-conjugated CRM (F₁-CRM), or with F₁-CRM in combination with varying quantities of alum, TLR4 agonist (the compound of Formula V), and TLR7/8 agonist (the compound of Formula IV). Balb/c mice were immunized twice by injection 14 days apart with either unadjuvanted F₁-CRM or adjuvanted F₁-CRM, after which serum was collected and analyzed for antibodies against fentanyl 14 days after the second injection (n=8 for all groups). FIG. 3A indicates total IgG titers. FIG. 3B indicates IgG1 titers. FIG. 3C indicates IgG2a titers. Asterisks indicate statistical significance compared to F₁-CRM alone (*: p<0.05; **: p<0.01; ***: p<0.01; ****: p<0.0001).

FIGS. 4A to 4D show the efficacy of adjuvanted fentanyl vaccines compared to non-adjuvanted fentanyl vaccines in a murine model. FIG. 4A shows fentanyl-specific serum IgG titers produced after 21 days in response to immunization with unconjugated CRM (CRM), fentanyl-conjugated CRM (F₁-CRM), or F₁-CRM in combination with alum and TLR4 agonist (the compound of Formula V) and TLR7/8 agonist (the compound of Formula IV). FIG. 4B show fentanyl-specific serum IgG titers as in FIG. 4A, but after 32 days. FIG. 4C shows respiratory depression in mice immunized as in FIG. 4A and FIG. 4B 30 minutes after being challenged with 0.05 mg/kg fentanyl. FIG. 4D shows antinociception in mice immunized as in FIG. 4A and FIG. 4B 30 minutes after being challenged with 0.05 mg/kg fentanyl, as measured with a hot plate test. All data in FIGS. 4A to 4D reflect means±SEM, n=6-7 per group. Significance is indicated by asterisks (*: p<0.05; **: p<0.01; ***: p<0.01; ****: p<0.0001). Significance in FIGS. 4A and 4B is shown in relation to the F₁-CRM group. Significance in FIGS. 4C and 4D is shown in relation to the unconjugated CRM group.

FIGS. 5A to 5C show responses to fentanyl challenge in mice 21 days after vaccination. Mice were vaccinated with CRM alone, F₁-CRM, or F₁-CRM with different adjuvants and challenged with 0.05 mg/kg fentanyl 21 days after the first vaccination. FIG. 5A shows fentanyl-specific serum IgG titers after two immunizations. FIG. 5B shows fentanyl-induced antinociception as a percent of the maximum possible effect (MPE %), as measured by a hot plate test 30 min. after drug challenge. FIG. 5C shows the percent change in heart rate as compared to baseline, measured via pulse oximetry 30 min. after drug challenge. All data reflect means±SEM, n=6-7 per group. Significance is indicated by asterisks (*: p<0.05; **: p<0.01; ***: p<0.01; ****: p<0.0001).

FIGS. 6A to 6F show responses to fentanyl challenge in mice 35 days after vaccination. Mice were re-challenged at 35 days with 0.05 mg/kg fentanyl, using a similar paradigm as the 21 day challenge. FIG. 6A shows fentanyl-specific serum IgG titers at 34 days. FIG. 6B shows the ratio of IgG2a to IgG1 subclasses at 34 days. FIG. 6C shows the percent change in heart rate 30 min. after drug challenge, measured via collar pulse oximeter. FIG. 6D shows fentanyl-induced antinociception as a percent of the maximum possible effect (MPE %) 30 minutes after drug challenge, measured by hot plate test. FIG. 6E and FIG. 6F show the concentration of fentanyl in the serum and brain, respectively, 30 min. after drug challenge, measured via LC-MS. All data reflect means±SEM, n=5-6 per group. Asterisks directly over columns indicate significance compared to control (*: p<0.05; **: p<0.01; ***: p<0.01; ****: p<0.0001). Brackets indicate specific group comparisons, and “#” over columns indicates significance to F₁-CRM.

FIGS. 7A to 7C show analyses of antigen-specific B cells via flow cytometry 7 days after a single vaccination. Mice were euthanized 7 days after a single immunization, and spleens and lymph nodes were processed for flow cytometry. FIG. 7A shows the total number of B cells per sample. FIG. 7B shows the number of fentanyl-specific B cells per sample. FIG. 7C shows the number of fentanyl-specific B cells displaying switched immunoglobulin Fc regions. All data reflect means±SEM, n=2-3 per group. Asterisks directly over columns indicate significance compared to control (*: p<0.05; **: p<0.01; ***: p<0.01; ****: p<0.0001). Brackets indicate specific group comparisons, and “#” over columns indicate significance to F₁-CRM.

FIGS. 8A to 8D show responses to an acute fentanyl dose (0.05 mg/kg) in rats. After immunization with CRM alone, F₁-CRM, or F₁-CRM with various adjuvants, FIG. 8A shows fentanyl-specific serum IgG titers as measured via ELISA at 49 days. On day 56, rats were challenged with fentanyl. FIG. 8B and FIG. 8C show the concentration of fentanyl in the serum and brain, respectively, 30 min. after drug challenge, measured via LC-MS. FIG. 8D shows response latency as measured via hot plate test 15 min. after drug challenge. FIG. 8E, and FIG. 8F show heart rate and oxygen saturation, respectively, as measured using a pulse oximeter 30 min. after drug challenge. All data reflect means±SEM, n=2-3 per group. Asterisks directly over columns indicate significance compared to control (*: p<0.05; **: p<0.01; ***: p<0.01; ****: p<0.0001). Brackets indicate specific group comparisons, and “#” over columns indicate significance to F₁-CRM.

FIGS. 9A to 9C show responses to a cumulative dosing paradigm in rats. After immunization, rats were challenged with 0.05 mg/kg fentanyl every 15 min. until a cumulative dose of 0.45 mg/kg fentanyl was reached. FIG. 9A shows fentanyl-specific serum IgG titers measured one week before drug challenge. FIG. 9B shows a survival curve indicating the number of rats whose oxygen saturation dropped below 50% during the cumulative dosing paradigm, at which point they were removed from the study and rescued by administration of naloxone. FIG. 9C shows oxygen saturation over increasing cumulative fentanyl dose, which was used to calculate an ED₅₀ for each treatment group. Data displayed in FIGS. 9A and 9C reflect means±SEM, n=4-5 per group. Asterisks directly over columns indicate significance compared to control (*: p<0.05; **: p<0.01; ***: p<0.01; ****: p<0.0001). Brackets indicate specific group comparisons.

FIGS. 10A to 10E show responses to an acute fentanyl dose (0.1 mg/kg) in rats previously administered a cumulative dosing paradigm. After a cumulative dosing challenge, rats were given a washout period of one week before they were challenged with an acute dose of 0.1 mg/kg fentanyl. FIG. 10A shows fentanyl-induced antinociception as a percentage of maximum possible effect (% MPE), measured via a hot plate test 30 min. after drug challenge. FIG. 10B and FIG. 10C show oxygen saturation and heart rate, respectively, measured via collar pulse oximeter 30 min. after drug challenge. FIG. 10D shows a linear regression of oxygen saturation plotted against fentanyl-specific serum IgG titers. FIG. 10E shows a linear regression of hot plate response latency plotted against fentanyl-specific serum IgG titers. Data displayed in FIG. 10A to 10C reflect means±SEM, n=5-6 per group. Asterisks directly over columns indicate significance compared to control (*: p<0.05; **: p<0.01; ***: p<0.01; ****: p<0.0001). Brackets indicate specific group comparisons.

### DETAILED DESCRIPTION OF CERTAIN EMBODIMENTS

Some aspects of the present disclosure provide vaccines comprising an opioid antigen and a toll-like receptor 7 and 8 (TLR7/8) agonist. The TLR7/8 agonist is used in the FEN vaccine as an adjuvant. The TLR7/8 agonist may be formulated with an opioid vaccine for treating OUD.

Accordingly, some aspects of the present disclosure provide methods of treating an opioid use disorder (OUD) in a subject in need thereof. In some embodiments, the method comprises administering to the subject an effective amount of a composition comprising a toll-like receptor 7 and 8 (TLR7/8) agonist. In some embodiments, the method comprises administering to the subject an effective amount of a composition comprising an opioid antigen and a toll-like receptor 7 and 8 (TLR7/8) agonist.

An “opioid antigen” refers to a molecule that can induce immune response to opioid. Examples of opioid antigens include, without limitation, fentanyl, heroin, hydromorphone, 6-acetylmorphine, morphine, oxycodone, hydrocodone, codeine, or an analog thereof.

In some embodiments, the opioid antigen is a fentanyl (FEN). A “fentanyl (FEN)” refers to a powerful synthetic opioid analgesic that is similar to morphine but is 50 to 100 times more potent. It is a Schedule II prescription drug, and it is typically used to treat patients with severe pain or to manage pain after surgery. It is also sometimes used to treat patients with chronic pain who are physically tolerant to other opioids. In some embodiments, fentanyl is used as the antigen in the compositions described herein. In some embodiments, the FEN antigen is a fentanyl (FEN)-based hapten, (e.g., FEN is partially modified by an additional chemical group that enables its conjugation to a carrier that enhances its immunogenicity). FEN-based hapten is also referred to as F₁ herein. FEN-based hapten has been described, e.g., in Ealeign et al., J Pharmacol Exp Ther. 2019 February; 368(2): 282-291, incorporated herein by reference. Structures of FEN and FEN based hapten are shown below.

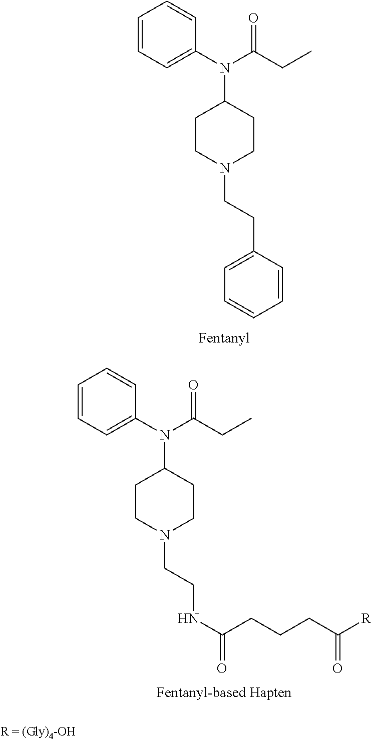

In some embodiments, the opioid antigen (e.g., FEN or FEN-based hapten) is conjugated to a carrier protein. Non-limiting examples of carrier proteins that may be conjugated to the opioid antigen described herein include tetanus toxoid (TT), detoxified cross-reactive material (CRM) from diphtheria toxin, keyhole limpet hemocyanin (KLH), subunit KLH (sKLH), meningococcal outer membrane protein complex (OMPC), and H. influenzae protein D (HiD).

Toll-like receptor 7 and 8 (TLR7/8) belong to the TLR family that plays an important role in pathogen recognition and activation of innate immunity. TLRs are highly conserved and share structural and functional similarities. When microbes breach the body's physical barriers, they are recognized by TLRs expressed on the membranes of leukocytes, which include dendritic cells, macrophages, natural killer cells, T cells, B cells, and non-immune cells. TLRs recognize pathogen-associated molecular patterns (PAMPs) that are expressed on infectious microorganism and mediate the production of cytokines necessary for effitive immunity by regulating immune activation, inflammation, survival, and proliferation. At least six TLR family members are located on the cell surface (TLR1, TLR2, TLR4, TLR5, TLR6, and TLR11), while four are located on lysosomal and endosomal surface (TLR1, TLR2, TLR4, TLR5, TLR6, and TLR11). The various TLRs exhibit different patterns of expression across tissues. TLR7 is predominantly expressed in lung, placenta, and spleen, and lies in close genetic proximity to TLR8 on the human X chromosome. TLR7/8 are involved in the response to viral infection. They recognize single-stranded RNAs as well as small synthetic molecules as their natural ligands.

An “agonist” is a chemical that binds to a receptor and activates the receptor to produce a biological response. TLR7/8 agonists activate the signaling pathway mediated by TLR7/8 and have been described, e.g., in Dowling et al., ImmunoHorizons Jul. 1, 2018, 2 (6) 185-197, incorporated herein by reference. Known TLR7/8 agonists include, without limitation, CL075 (a thiazoloquinoline compound), CL097 (an imidazoquinoline compound), and R848 (an imidazoquinoline compound).

In some embodiments, the composition comprising the FEN antigen and the TLR7/8 agonist described herein is immunogenic. In some embodiments, the composition comprising the FEN antigen and the TLR7/8 is an opioid vaccine. Being “immunogenic” means that the composition elicits immune response when administered to a subject (e.g., a mammalian subject such as a human). As used herein, an “immune response” refers to a response by a cell of the immune system, such as an antigen-presenting cell, dendritic cell, monocyte, macrophage, NKT cell, NK cell, basophil, eosinophil, or neutrophil, B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an antigen or an adjuvant).

In some embodiments, the immune response elicited by the composition (e.g., opiod vaccine) described herein is specific for a particular antigen (an “antigen-specific response” or “adaptive immune response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

In some embodiments, an antigen-specific immune response includes both a humoral and/or a cell-mediated immune response to the antigen. A “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art (e.g., Erickson et al. (1993) J. Immunol. 151:4189-4199; and Doe et al. (1994) Eur. J. Immunol. 24:2369-2376).

In some embodiments, the immune response elicited by the composition (e.g., opiod vaccine) described herein is an innate immune response. An “innate immune response” refers to the response by the innate immune system. The innate immune system uses a set of germline-encoded receptors (“pattern recognition receptor” or “PRR”) for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. In some embodiments, the innate immune response elicited by the composition (e.g., opiod vaccine) described herein confers heterologous (“non-specific”) immunity to a broad range of pathogenic microbes by enhancing innate immune responses to subsequent stimuli, a phenomenon known as “trained immunity”, a form of innate memory, e.g., as described in Netea et al. (Trained Immunity: An Ancient Way of Remembering. Cell Host Microbe. 2017 Mar. 8; 21(3):297-300, incorporated herein by reference).

The receptors of the innate immune system that recognize PAMPs are called Pattern Recognition Receptors (PRRs). (Janeway et al. (1989) Cold Spring Harb. Symp. Quant. Biol. 54: 1-13; Medzhitov et al. (1997) Curr. Opin. Immunol. 94: 4-9, incorporated herein by reference). PRRs vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3, incorporated herein by reference). Non-limiting examples of PRRs include: toll-like receptors (e.g., TLR2), NOD1/2, RIG-1/MDA-5, C-type lectins, and STING.

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, including, without limitation: chemokines, interferons, interleukins, lymphokines, and tumour necrosis factors. This latter function allows efficient mobilization of effector forces to combat the invaders.

In some embodiments, the composition described herein is a vaccine composition (e.g., opiod vaccine). The terms “vaccine composition” and “vaccine” are used interchangeably herein. A “vaccine composition” is a composition that activates or enhances a subject's immune response to an antigen after the vaccine is administered to the subject. In some embodiments, a vaccine stimulates the subject's immune system to recognize the antigen (e.g., FEN antigen) as foreign, and enhances the subject's immune response if the subject is later exposed to the pathogen, whether attenuated, inactivated, killed, or not. Vaccines may be prophylactic, for example, preventing or ameliorating a detrimental effect of a future exposure to a pathogen, or therapeutic, for example, activating the subject's immune response to an antigen (e.g., FEN antibody) after the subject has been exposed to the antigen (e.g., FEN antigen). In some embodiments, a vaccine composition is used to protect against or treat opioid use disorder (OUD).

In some embodiments, the TLR7/8 agonist described herein is used as an adjuvant in the composition (e.g., opioid vaccine) described herein. An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system (e.g., H. S. Warren et al., (1986) Annu. Rev. Immunol., 4:369, incorporated herein by reference). The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or derivatives such as monophosphoryl lipid A (MPLA) and glycopyranosyl lipid A (GLA), molecular cages for antigen, components of bacterial cell walls, endocytosed nucleic acids such as double-stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as aluminum hydroxide or alum. At present, currently licensed vaccines in the United states contain only a limited number of adjuvants, such as alum that enhances production of TH 2 cells are and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Guérin (BCG); microorganism components, e.g., alum-precipitated diphtheria toxoid, bacterial lipopolysaccharides (“endotoxins”) and their derivatives such as MPLA and GLA.

In some embodiments, the TLR7/8 agonist used in the composition (e.g., opioid vaccine) described herein is an imidazoquinoline compound. An “imidazoquinoline compound” is a compound bearing the core structure of imidazoquinoline as shown of Formula I. In some embodiments, the molecule is further modified at positions R¹ and R². In some embodiments, R¹ is selected from chemical groups containing a phospholipid, lipid, lipidation and/or PEG. In some embodiments, R² is selected from H, alkyl, alkylamino, alkoxy, cycloalkyl, alkylamino groups that are unbranched or branched and optionally terminally substituted with a hydroxyl, amino, thio, hydrazino, hydrazido, azido, acetylenyl, carbonyl, or maleimido group.

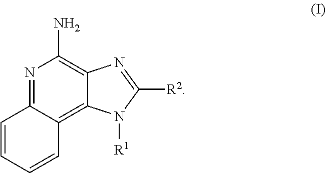

In some embodiments, the TLR7/8 agonist used in the composition (e.g., opioid vaccine) described herein is R848, also known as resiquimod, as described in U.S. Pat. No. 5,395,937. which is of Formula II and is incorporated herein by reference:

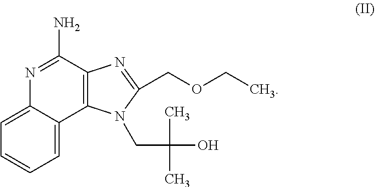 [SMILES: CCOCc1nc2c(N)nc3ccccc3c2n1CC(C)(C)O]

In some embodiments, the TLR7/8 agonist used in the composition (e.g., opioid vaccine) described herein is an oxoadenine compound. An “oxoadenine compound” is a compound bearing the core structure of 8-oxoadenine as depicted in Formula III, wherein the molecule is further modified at positions R¹ and R².

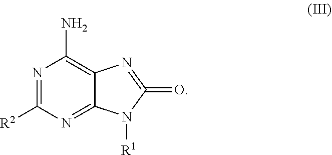

In some embodiments, the TLR7/8 agonist used in the composition (e.g., opioid vaccine) described herein is of Formula IV:

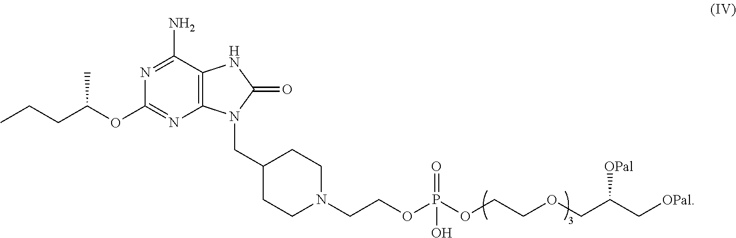

In some embodiments, the composition (e.g., opioid vaccine) described herein comprises more than one TLR7/8 agonists (e.g., 1, 2, 3, or more) described herein. In some embodiments, the composition described herein comprises an imidazoquinoline compound (e.g., the compound of Formula II) and an oxoadenine compound (e.g., the compound of Formula IV).

In some embodiments, the composition (e.g., opioid vaccine) described herein further comprises a second adjuvant, in addition to the TLR7/8 agonist (as the first adjuvant). Any adjuvants known in the art or described herein may be used as the second adjuvant in the composition. In some embodiments, the second adjuvant is an agonist of Pattern Recognition Receptors (PRRs) such as Toll-like receptors (TLRs), NOD-like receptors (NLRs), RIG-I-like receptor (RLR), C-type Lectin receptors (CLRs), and a stimulator of interferon genes (STING). Agonists of the PPRs enhance immune responses (e.g., innate or adaptive immune response). Agonists of PPRs are known to those skilled in the art. For example, various TLR and NLR agonists are described in Kaczanowska et al., (2013) J Leukoc Biol 93(6): 847-863; Higgins et al., (2010) Curr Infect Dis Rep. 12(1):4-12; and Maisonneuve et al., (2014) Proc Natl Acad Sci USA.; 111(34): 12294-12299, incorporated herein by reference. RIG-I-like receptor agonists are described in Ranjith-Kumar et al., (2009) J Biol Chem.; 284(2): 1155-1165; and Goulet et al., PLOS Pathogens 9(8): 10, incorporated herein by reference. CLR agonists are described in Lamb et al., (2002) Biochemistry 41(48):14340-7; and Yan et al., (2015) Front Immunol. 6: 408, incorporated herein by reference. STING agonists are described in Fu et al., (2015) Sci Transl Med. 7(283): 283ra52; and Foote et al., Cancer Immunology Research, DOI: 10.1158/2326-6066.CIR-16-0284, incorporated herein by reference. The PRR agonists described herein are also commercially available, e.g., from InvivoGen (California, USA). In some embodiments, the second adjuvant is alum.

In some embodiments, the composition (e.g., opioid vaccine) described herein further comprises a TLR4 agonist (e.g., as a second adjuvant). In some embodiments, the TLR4 agonist is of Formula V:

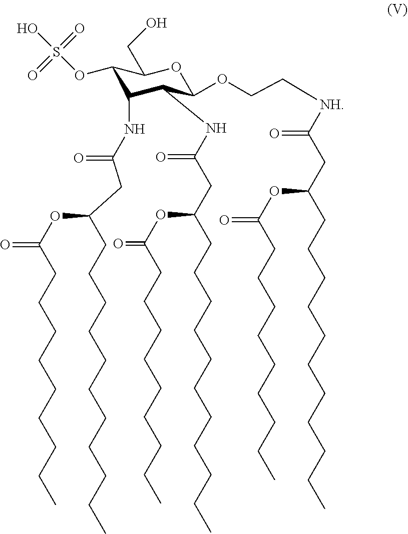

In some embodiments, any one of the TLR7/8 agonists (e.g., imidazoquinoline compounds or oxoadenine compounds) and/or TLR4 agonists used in the compositions (e.g., opioid vaccines) described herein are lipidated (e.g., with PEG). In some embodiments, the composition (e.g., opioid vaccine) described herein further comprises alum. In some embodiments, the TLR7/8 and/or TLR4 agonist is adsorbed in the alum in the composition (e.g., opioid vaccine) described herein.

In some embodiments, the composition (e.g., opioid vaccine) described herein are formulated for administration to a subject. In some embodiments, the composition (e.g., opioid vaccine) is formulated or administered in combination with one or more pharmaceutically acceptable excipients. In some embodiments, compositions (e.g., opioid vaccines) comprise at least one additional active substance, such as, for example, a therapeutically active substance, a prophylactically-active substance, or a combination of both. The compositions described herein may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

Formulations of the compositions (e.g., opioid vaccines) described herein may be prepared by any method known or hereafter developed in the art of pharmacology. In general, such preparatory methods include the step of bringing the antigen and/or the adjuvant (e.g., TLR7/8 agonists) into association with an excipient and/or one or more other accessory ingredients, and then, if necessary and/or desirable, dividing, shaping and/or packaging the product into a desired single- or multi-dose unit.

Relative amounts of the antigen (e.g., the FEN antigen), the adjuvant (e.g., the TLR7/8 agonist), the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the composition (e.g., opioid vaccine) described herein are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, the composition (e.g., opioid vaccine) is formulated in an aqueous solution. In some embodiments, the composition (e.g., opioid vaccine) is formulated in a nanoparticle. In some embodiments, the composition (e.g., opioid vaccine) is formulated in a lipid nanoparticle. In some embodiments, the composition (e.g., opioid vaccine) is formulated in a lipid-polycation complex, referred to as a lipid nanoparticle. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is incorporated herein by reference. In some embodiments, the composition (e.g., opioid vaccine) is formulated in a lipid nanoparticle that includes a non-cationic lipid such as, but not limited to, cholesterol or dioleoyl phosphatidylethanolamine (DOPE).

In some embodiments, the methods described herein are for treating a human subject having an opioid use disorder (OUD). An “opioid use disorder (OUD)” is any of a set of disorders that can result from the prescribed use of opioid medications, misuse of prescribed opioid medications, use of diverted opioid medications, or use of illicitly obtained opioids such as heroin. OUD is typically a chronic, relapsing illness, associated with significantly increased rates of morbidity and mortality. Subjects with OUD experience a problematic pattern of opioid use and can exhibit symptoms of opioid withdrawal. In some embodiments, the OUD is opioid addition. In some embodiments, the OUD is opioid tolerance. In some embodiments, the OUD is opioid overdose.

In some embodiments, the subject is a human adolescent. In some embodiments, the subject is a human adult. In some embodiments, the subject is a human that is between ages 18-30 (e.g., 18-30, 18-25, 18-20, 20-30, 20-25, or 25-30 years old).

The subject treated using the methods described herein have used, are addicted to, or are prone to the use of opioids. “Opioids” and “opiates” refer to any of a set of natural or synthetic organic substances that act on opioid receptors and are used to reduce acute and chronic pain in a subject. “Addiction” refers to a chemical dependence that is established in a subject following use of an addictive substance such as an opioid, especially where such use was prolonged and/or frequent. In some embodiments, the opioid is heroin. In some embodiments, the opioid is a synthetic opioid, such as fentanyl.

The FEN antigen and/or the TLR7/8 agonists described herein elicits an immune response in the subject. In some embodiments, the immune response is an innate immune response. In some embodiments, the immune response is an adaptive immune response specific to the antigen in the composition or vaccine. In some embodiments, the antigen and/or the TLR7/8 agonist activates B cell immunity. In some embodiments, the antigen and/or the TLR7/8 agonist elicits antibody production. In some embodiments, the composition or the vaccine activates cytotoxic T cells specific to the antigen.

In some embodiments, the TLR7/8 agonist, whether administered alone or in an admixture with the FEN antigen, enhance the innate immune response, compared to without the TLR7/8 agonist or when the FEN antigen is administered alone. In some embodiments, the TLR7/8 agonist activates peripheral blood mononuclear cells (PBMCs). In some embodiments, the number of PBMCs that are activated is increased by at least 20% in the presence of a TLR7/8 agonist, compared to without the TLR7/8 agonist or when the FEN antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of a TLR7/8 agonist, compared to without the TLR7/8 agonist or when the FEN antigen is administered alone. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more, in the presence of a TLR7/8 agonist, compared to without the TLR7/8 agonist or when the FEN antigen is administered alone.

In some embodiments, the TLR7/8 agonist activates a pattern recognition receptor (PRR). In some embodiments, the PRR is selected from the group consisting of Toll-like receptors (e.g., TLR2), NOD1/2, RIG-1/MDA-5, C-type lectins, and STING. In some embodiments, the TLR is TLR-1, -2, -3, -4, -5, -6, -9, -10. In some embodiments, the TLR is TLR-7 or -8. In some embodiments, the number of PRRs that are activated is increased by at least 20% in the presence of a TLR7/8 agonist, compared to without the TLR7/8 agonist or when the FEN antigen is administered alone. For example, the number of PRRs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of a TLR7/8 agonist, compared to without the TLR7/8 agonist or when the FEN antigen is administered alone. In some embodiments, the number of PRRs that are activated is increased by 20%, 30%, 40%, 50%, 60%, 70%, 80%, 90%, 100%, 2-fold, 5-fold, 10-fold, 100-fold, 1000-fold or more, in the presence of a TLR7/8 agonist, compared to without the TLR7/8 agonist or when the FEN antigen is administered alone.

In some embodiments, the TLR7/8 agonist induces the production of a proinflammatory cytokine (e.g., TNF, IL-12, IL-6, or IL1-β) and/or chemokines (e.g., CCL3) in the subject. In some embodiments, the level of proinflammatory cytokines and/or chemokines (e.g., CCL3) is increased by at least 20% in the presence of a TLR7/8 agonist, compared to without the TLR7/8 agonist or when the FEN antigen is administered alone. For example, the level of proinflammatory cytokines (e.g., TNF, IL-12, IL-6, or IL1-β) and/or chemokines (e.g., CCL3) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of a TLR7/8 agonist, compared to without the TLR7/8 agonist or when the FEN antigen is administered alone. In some embodiments, the level of proinflammatory cytokines (e.g., TNF, IL-12, IL-6, or IL1-β) and/or chemokines (e.g., CCL3) is increased by 20%, 30%, 40%, 50%, 60%, 70%, 80%, 90%, 100%, 2-fold, 5-fold, 10-fold, 100-fold, 1000-fold or more, in the presence of a TLR7/8 agonist, compared to without the TLR7/8 agonist or when the FEN antigen is administered alone.

In some embodiments, the TLR7/8 agonist enhances innate immune memory (also referred to as trained immunity). “Innate immune memory” confers heterologous immunity that provides broad protection against a range of pathogens. In some embodiments, the innate immune memory is increased by at least 20% in the presence of a TLR7/8 agonist, compared to without TLR7/8 agonist or when the FEN antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of a TLR7/8 agonist, compared to without the TLR7/8 agonist or when the FEN antigen is administered alone. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of a TLR7/8 agonist, compared to without the TLR7/8 agonist or when the FEN antigen is administered alone.

In some embodiments, the TLR7/8 agonist, when administered as an admixture with an antigen (e.g., the vaccine composition described herein), enhances the anti-specific immune response against the antigen or against the invading agent where the antigen is derived from (e.g., a microbial pathogen or cancer), compared to without the TLR7/8 agonist, i.e., when the FEN antigen is administered alone. In some embodiments, the TLR7/8 agonist enhances the production of antigen-specific antibody titer (e.g., by at least 20%) in the subject, compared to without the TLR7/8 agonist, i.e., when the FEN antigen is administered alone. For example, the TLR7/8 agonist may enhance the production of antigen-specific antibody titer by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the TLR7/8 agonist, i.e., when the FEN antigen is administered alone. In some embodiments, the TLR7/8 agonist enhances the production of antigen-specific antibody titer by 20%, 30%, 40%, 50%, 60%, 70%, 80%, 90%, 100%, 2-fold, 5-fold, 10-fold, 100-fold, 1000-fold or more, in the presence of a TLR7/8 agonist, compared to without the TLR7/8 agonist, i.e., when the FEN antigen is administered alone. One skilled in the art is familiar with how to evaluate the level of an antibody titer, e.g., by ELISA.

In some embodiments, the TLR7/8 agonist enhances the activation of cytotoxic T-cells (e.g., by at least 20%) in the subject, compared to without the TLR7/8 agonist, i.e., when the FEN antigen is administered alone. For example, the TLR7/8 agonist may enhance activation of cytotoxic T-cells by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the subject, compared to without TLR7/8 agonist, i.e., when the FEN antigen is administered alone. In some embodiments, the TLR7/8 agonist enhances the activation of cytotoxic T-cells by 20%, 30%, 40%, 50%, 60%, 70%, 80%, 90%, 100%, 2-fold, 5-fold, 10-fold, 100-fold, 1000-fold or more, compared to without the TLR7/8 agonist, i.e., when the FEN antigen is administered alone.

It has been demonstrated that the innate immune system plays a crucial role in the control of initiation of the adaptive immune response and in the induction of appropriate cell effector responses. (Fearon et al. (1996) Science 272: 50-3; Medzhitov et al. (1997) Cell 91: 295-8, incorporated herein by reference). As such, in some embodiments, the TLR7/8 agonist enhances the innate immune response in a subject (e.g., when administered alone or in an admixture with an antigen), which in turn enhances the adaptive immune response against the antigen in the subject. This is particular useful in subjects that have undeveloped (e.g., in an neonatal infant), weak (e.g., in an elderly), or compromised immune systems (e.g., in a patient with primary immunodeficiency or acquired immunodeficiency secondary to HIV patient infection or a cancer patient undergoing with or without chemotherapy and/or radiation therapy).

In some embodiments, the TLR7/8 agonist prolongs the effect of a vaccine (e.g., by at least 20%) in the subject, compared to without the TLR7/8 agonist (i.e., when the FEN antigen is administered alone). For example, the TLR7/8 agonist may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the subject, compared to without the TLR7/8 agonist, i.e., when the FEN antigen is administered alone. In some embodiments, the TLR7/8 agonist prolongs the effect of a vaccine by 20%, 30%, 40%, 50%, 60%, 70%, 80%, 90%, 100%, 2-fold, 5-fold, 10-fold, 100-fold, 1000-fold or more, compared to without the TLR7/8 agonist, i.e., when the FEN antigen is administered alone.

In some embodiments, the TLR7/8 agonist increases rate of (accelerates) an immune response, compared to without the TLR7/8 agonist, i.e., when the FEN antigen is administered alone. For example, the TLR7/8 agonist may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the TLR7/8 agonist, i.e., when the FEN antigen is administered alone. In some embodiments, the TLR7/8 agonist increases the rate of an immune response by 20%, 30%, 40%, 50%, 60%, 70%, 80%, 90%, 100%, 2-fold, 5-fold, 10-fold, 100-fold, 1000-fold or more, compared to without the TLR7/8 agonist, i.e., when the FEN antigen is administered alone. “Increase the rate of immune response” mean it takes less time for the immune system of a subject to react to an invading agent (e.g., a microbial pathogen).

In some embodiments, the antigen produces a same level of immune response against the antigen at a lower dose in the presence of the TLR7/8 agonist, compared to without the TLR7/8 agonist, i.e., when the FEN antigen is administered alone. In some embodiments, the amount of antigen needed to produce the same level of immune response is reduced by at least 20% in the presence of the TLR7/8 agonist, compared to without the TLR7/8 agonist, i.e., when the FEN antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more, in the presence of the TLR7/8 agonist, compared to without the TLR7/8 agonist, i.e., when the FEN antigen is administered alone. In some embodiments, the amount of antigen needed to produce the same level of immune response is reduced by at 20%, 30%, 40%, 50%, 60%, 70%, 80%, 90%, 95%, 99% or more, in the presence of the TLR7/8 agonist, compared to without the TLR7/8 agonist, i.e., when the FEN antigen is administered alone.

The prophylactic or therapeutic use of the TLR7/8 agonist, or the composition or vaccine composition described herein is also within the scope of the present disclosure. In some embodiments, the composition or vaccine composition described herein are used in methods of vaccinating a subject by prophylactically administering to the subject an effective amount of the composition or vaccine composition described herein. “Vaccinating a subject” refer to a process of administering an immunogen, typically an antigen formulated into a vaccine, to the subject in an amount effective to increase or activate an immune response against the antigen and, thus, against a pathogen displaying the antigen. In some embodiments, the terms do not require the creation of complete immunity against the pathogen. In some embodiments, the terms encompass a clinically favorable enhancement of an immune response toward the antigen or pathogen. Methods for immunization, including formulation of a vaccine composition and selection of doses, routes of administration and the schedule of administration (e.g. primary dose and one or more booster doses), are well known in the art. In some embodiments, vaccinating a subject reduces the risk of developing a disease (e.g., OUD) in a subject.

In some embodiments, the composition or vaccine composition described herein are formulated for administration to a subject. In some embodiments, the composition or vaccine composition further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the composition or vaccine composition described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The composition or vaccine composition described herein may conveniently be presented in unit dosage form and may be prepared by any of the methods well-known in the art of pharmacy. The term “unit dose” when used in reference to a composition or vaccine composition described herein of the present disclosure refers to physically discrete units suitable as unitary dosage for the subject, each unit containing a predetermined quantity of active material calculated to produce the desired therapeutic effect in association with the required diluent; i.e., carrier, or vehicle. Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In some embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. The formulation of the composition or vaccine composition described herein may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

For topical administration, the composition or vaccine composition described herein can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the composition or vaccine composition described herein used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or vaccine composition described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

In some embodiments, the present disclosure contemplates a vaccine composition comprising a pharmaceutically acceptable injectable vehicle. The vaccines of the present disclosure may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water soluble carriers such as muramyl dipeptides have been used.

Preparation of injectable vaccines of the present disclosure, includes mixing the antigen and/or the TLR7/8 agonists with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating vaccine compositions are well-known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., OUD) in a subject in need thereof. In certain embodiments, the kits are useful for preventing a disease (e.g., OUD) in a subject in need thereof. In certain embodiments, the kits are useful as enhancers of an immune response (e.g., innate and/or adaptive immune response), and/or adjuvants in a vaccine for a disease, (e.g., OUD) in a subject, biological sample, tissue, or cell.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., OUD) in a subject in need thereof. In certain embodiments, the kits and instructions provide for preventing a disease (e.g., OUD) in a subject in need thereof. In certain embodiments, the kits and instructions provide for enhancing of an immune response (e.g., innate and/or adaptive immune response) in a subject, biological sample, tissue, or cell. In certain embodiments, the kits and instructions provide for use of the compounds as adjuvants in a vaccine for a disease, (e.g., OUD) in a subject, biological sample, tissue, or cell. A kit described herein may include one or more additional pharmaceutical agents described herein as a separate composition.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The composition of the vaccine composition described herein may be administered systemically (e.g., via intravenous injection) or locally (e.g., via local injection). In some embodiments, the composition of the vaccine composition described herein is administered orally, intravenously, topically, intranasally, or sublingually. Parenteral administrating is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques. In some embodiments, the administering is done intramuscularly, intradermally, orally, intravenously, topically, intranasally, intravaginally, or sublingually. In some embodiments, the composition is administered prophylactically.

In some embodiments, the composition or vaccine composition is administered once or administered repeatedly (e.g., 2, 3, 4, 5, or more times). For multiple administrations, the administrations may be done over a period of time (e.g., 6 months, a year, 2 years, 5 years, 10 years, or longer). In some embodiments, the composition or vaccine composition is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later).

A “subject” to which administration is contemplated refers to a human (i.e., male or female of any age group, e.g., pediatric subject (e.g., infant, child, or adolescent) or adult subject (e.g., young adult, middle-aged adult, or senior adult) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or genetically engineered animal. A “subject in need thereof” refers to a human subject in need of treatment of OUD or in need of reducing the risk of developing OUD. In some embodiments, administering the antigen and TLR7/8 agonist described herein to a subject having OUD (therapeutic use). In some embodiments, administering the antigen and the TLR7/8 agonist described herein to a subject at risk of developing a disease reduces the likelihood (e.g., by 20%, 30%, 40%, 50%, 60%, 70%, 80%, 90%, 95%, 99% or more) of the subject developing OUD.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1

The opioid epidemic is a threat to public health. Adolescents and young adults (aged 18 to 30 years old) are at the epicenter of this crisis. Fentanyl (FEN) contamination of heroin (HER) and other illicit drugs has exacerbated the problem, increasing the rate of opioid overdose deaths among youth. A vaccine that specifically and effectively blocks FEN HER (or its metabolites—e.g., morphine (MOR) by inducing drug-specific antibodies (Ab) to form Ab-drug complex in the blood too large to cross the blood-brain barrier, could avert overdose deaths by preventing opioid binding to brainstem receptors that mediate respiratory suppression (Olson and Janda, (2018) EMBO Reports 19, 5-9). Hapten-based FEN vaccines, such as FEN conjugates to tetanus toxoid (FEN-TT) have been produced but demonstrate only modest Ab induction in animal models.

However, a prominent barrier to the utility of vaccines for opioid use disorder (OUD) is the variability in immunogenicity among individuals, who differ by age and known OUD history, which could be overcome via adjuvants. Additionally, limitations of animal models may be especially pronounced in comparison to patients with OUDs, as opioid use may impair an individual's immune response, posing additional challenges in development and pre-clinical evaluation of opioid vaccines. Indeed, efforts to date to develop opioid vaccines, whose protective effects are mediated by peripheral antibodies (Abs) that block penetration of opioids to the central nervous system thereby preventing opioid overdose death, have been hampered by weak and transient immunogenicity of opioid hapten vaccines. Notably, adjuvantation is a key approach to enhance vaccine-induced immunity. Adjuvants can enhance, prolong, and modulate immune responses to vaccinal antigens to maximize protective immunity, and may potentially enable effective immunization in vulnerable populations (e.g., individuals with altered immune responses).

In this context, the invention of a novel molecular approach to shape human immune responses in a distinct population is reported, i.e. individuals with a history of opioid use disorder, using small molecules with in vitro TLR7/8 receptor specific adjuvant activity towards human leukocytes. The activity of the small molecule TLR7/8 agonist R848 was tested to induce T-helper (Th) polarizing cytokine induction, using a whole blood assay. Blood was collected from individuals belonging to either a healthy control cohort or from participants with clinically diagnosed history of severe OUD recruited at Boston Children's Hospital Adolescent Substance Use & Addiction Program (ASAP).

In summary, as outlined below, it is reported that immune responses in youth with OUD may be distinct from their control healthy counterparts. This distinctness is exemplified by a reduced efficacy of the TLR4 agonist adjuvant MPLA in inducing innate cytokine responses in those with OUD. Specially, as compared to blood from control youth, blood of youth with OUD, upon stimulation with a range of stimuli, including Alum, MPLA and the Alum-adjuvanted hepatitis B vaccine Engerix, demonstrated reduced induction of cytokines and chemokines such as IL-12p40, IL-6, CXCL8, CXCL10, CCL2 and TNF. However, TLR7/8 agonist R848 induced a robust and balanced Th-polarizing cytokine production in whole blood from OUD as well as a control cohort (i.e., a non-inferior response).

This initial data suggests that novel adjuvant strategies may uniquely induce robust and balanced Th-polarizing cytokine production in whole blood from OUD participants that may lead to appropriately adjuvanted-opioid vaccines to induce immune responses favorable for enhancing protective immunity.

Overall, primary applications of the invention could include:
1) As stand-alone agent to modify human immune responses—e.g., since individuals with OUD may demonstrate distinct immune responses, small molecules TLR7/8 agonists could be applied topically to treat infections by enhancing an immune response; given orally to enhance mucosal immunity or intranasally to treat respiratory infection or to reduce allergy (e.g., allergic rhinitis); injected locally or systemically to enhance immune responses against tumors and cancers. Such a stand-alone formulation might also be given prophylactically to induce heightened immunity for broad protection against infection or radiation injury in populations with impaired immunity.
2) Adjunctive therapy to be given together with other treatments for the conditions listed above.
3) Vaccine adjuvants to be formulated with fentanyl-hapten or an alternate opioid antigen to enhance, accelerate, and/or broaden immune responses and/or to reduce the number of doses required (“dose sparing”), crucial given the costs of vaccinal antigens and challenges of multiple clinic visits when vaccine boosting is required to achieve protective immune responses.
   Advantages of the compositions and methods described herein include, at least:
1) Adjuvant activity in a distinct immune population with a history of OUD.
2) Small molecule category amenable to affordable scale up for mass production/use.
3) Molecular scaffold appears favorable from a medicinal chemistry perspective for practical and scalable production of congeners/analogues.
4) Activity towards human leukocytes.

### Methods

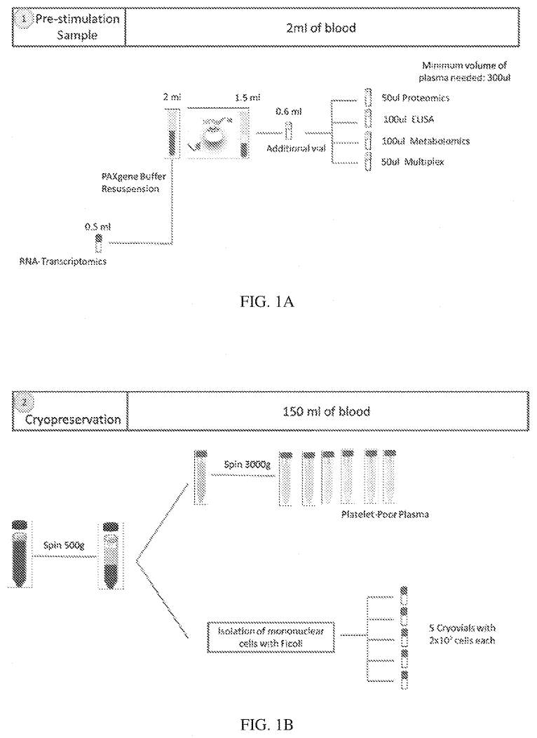

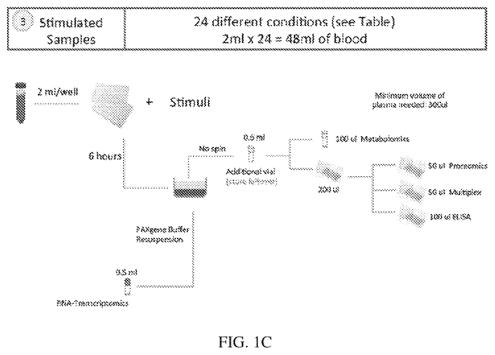

Blood samples were obtained from a pilot cohort of 15-30 year-old study participants enrolled in the Adolescent Substance use and Addiction program at Boston Children's Hospital (ASAP) and diagnosed with severe OUD as well as from healthy, age-matched controls (N=3 per group). A whole blood assay was employed in which heparinized blood in a 24-well plate format was stimulated with licensed adjuvanted and non-adjuvanted vaccines or with a FEN-tetanus toxoid (TT) hapten vaccine with or without candidate adjuvants (e.g., Alum, MPLA, R848, CpG) (FIGS. 1A-1C). After 6 hours, the extracellular medium was collected for multiplexed cytokine/chemokine analysis.

### Results

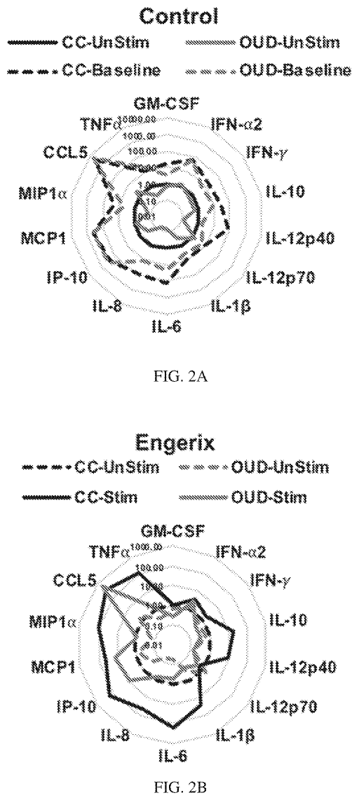

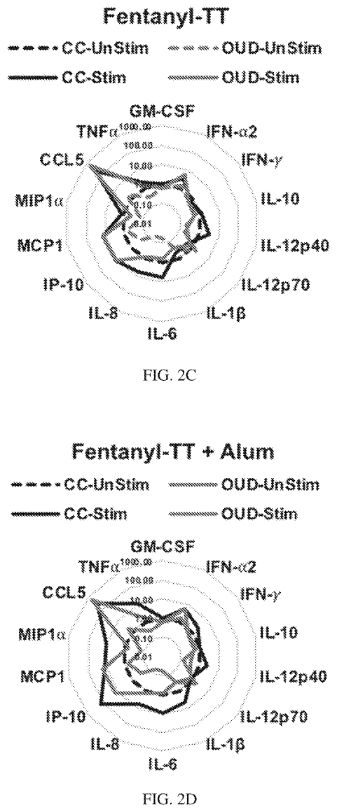

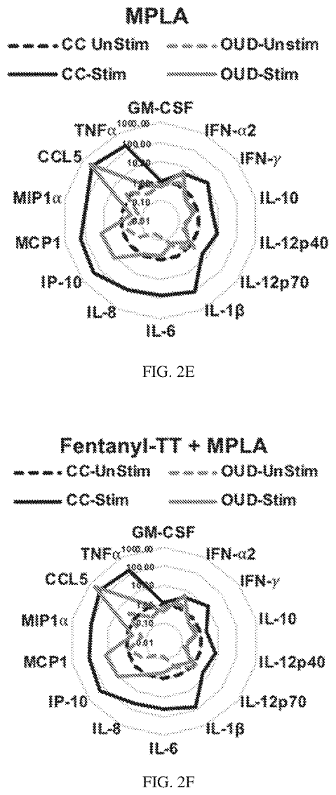

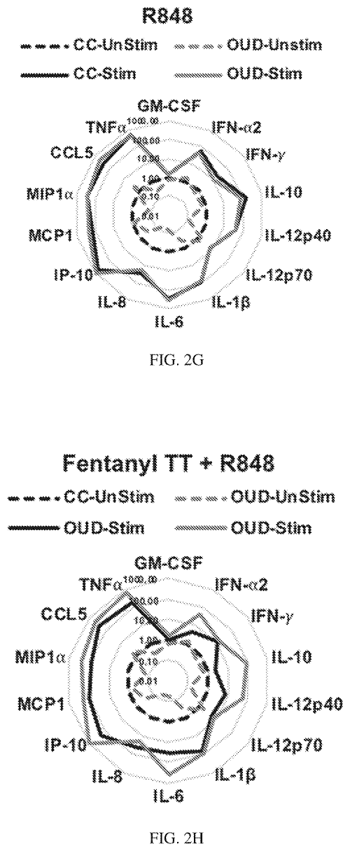

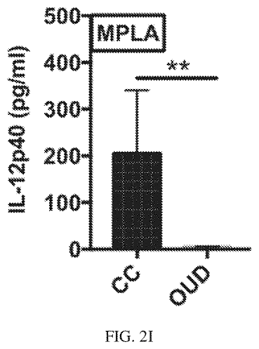

As compared to blood from control youth, blood of youth with OUD, upon stimulation with a range of stimuli, including Alum, MPLA and the Alum-adjuvanted hepatitis B vaccine Engerix, demonstrated impaired induction of cytokines and chemokines such as IL-12p40, IL-6, CXCL8, CXCL10, CCL2 and TNF. R848 induced a robust and balanced Th-polarizing cytokine production in whole blood from OUD as well as a control cohort (FIGS. 2A-2I).

### Conclusion

In this small pilot study, blood from youth with a history of OUD demonstrated reduced innate cytokine/chemokine responses to a range of candidate adjuvants in vitro reflecting a distinct immunophenotype that could affect their ability to generate robust opioid vaccine-induced Ab responses in vivo. Future studies will focus on increasing the number of study participants analyzed to further characterize opioid-induced impairment of immune function and the ability of certain candidate adjuvants to overcome the distinct OUD immunophenotype. Identification and optimization of adjuvantation systems tailored for youth with OUD may be key to advancing an effective opioid vaccine to avert opioid overdose deaths in this vulnerable population.

### Example 2

### Introduction

Previously small molecular agonists of TLR7/8 were shown to induce robust and balanced T-helper polarizing cytokine induction in whole blood from OUD subjects stimulated in vitro (Miller et al., Front. Immunol. (2020) 11:406). Concurrent with these in vitro studies, additional TLR4 and TLR7/8 agonist leads were evaluated in a series of in vivo experiments to determine the efficacy of hapten-carrier conjugate opioid vaccines adjuvanted with these agonists. These additional lead compounds included the compound of Formula IV, a lipidated oxoadenine ligand TLR7/8, as well as the compound of Formula V, a synthetic ligand of TLR4.

### Methods

To examine the efficacy of these additional agonists, Balb/c mice were injected with fentanyl-conjugated CRM antigens (F₁-CRM) either alone or adjuvanted with varying amounts of the aforementioned agonists and alum. After 14 days, mice were immunized a second time. After an additional 14 days, blood was collected from the mice from which serum was separated and analyzed for titers of immunoglobulin G (IgG). Changes in total IgG was determined, as well as changes in specific antibody subclasses. To further assess how these immunizations affected opioid sensitivity, certain groups of immunized mice were further challenged with fentanyl and examined for respiratory depression and antinociception.

### Results

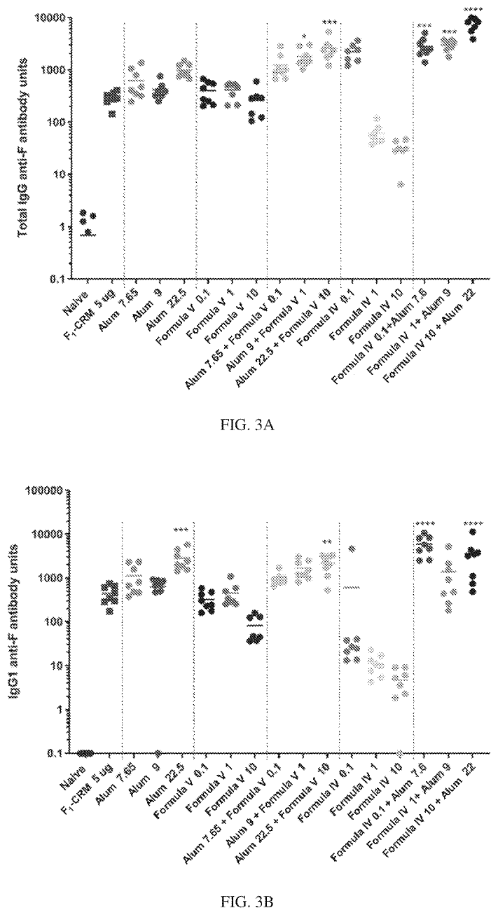

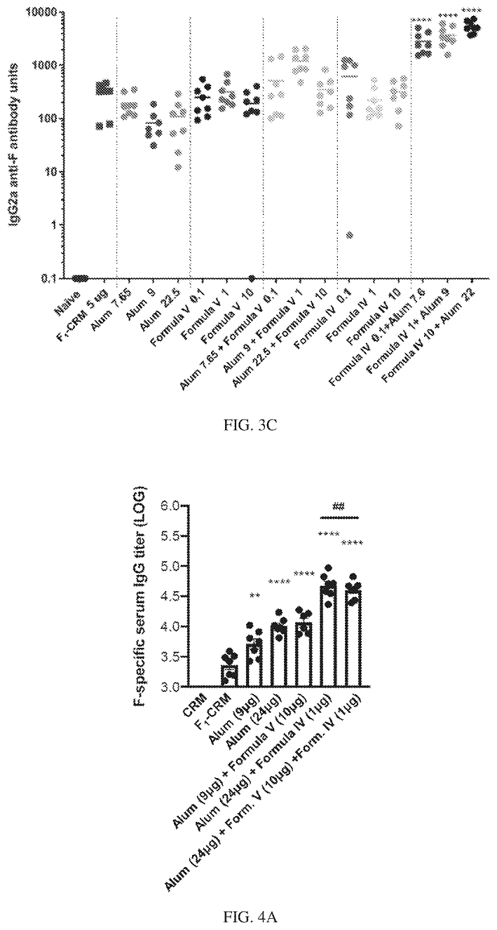

When administered F₁-CRM fentanyl conjugate antigen, both TLR4 agonist (e.g., the compound of Formula V) and TLR7/8 agonist (e.g., the compound of Formula IV) exhibited a dose responsive increase of anti-fentanyl antibody titers after two vaccinations when both adjuvants and antigen were adsorbed to alum (FIGS. 3A-3C). Strikingly, the TLR7/8 agonist (e.g., the compound of Formula IV) was such a strong Th1 polarizing adjuvant with F₁-CRM that serum IgG1 (Th2 indicating) titers were significantly reduced in mice vaccinated with F₁-CRM and the TLR7/8 agonist (e.g., the compound of Formula IV). This trend was reversed when the TLR7/8 agonist (e.g., the compound of Formula IV) was adsorbed to alum with F₁-CRM generating the highest IgG, IgG1 or IgG2a titers of any group.

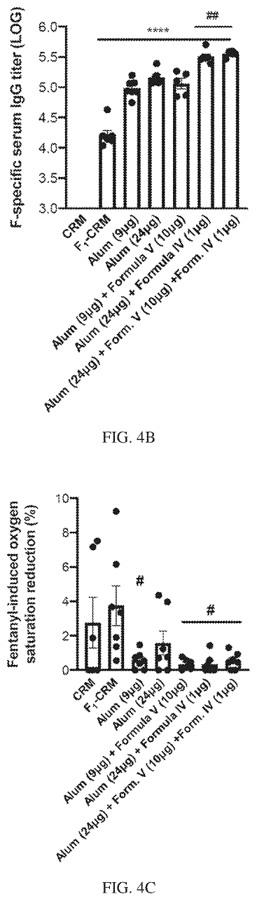

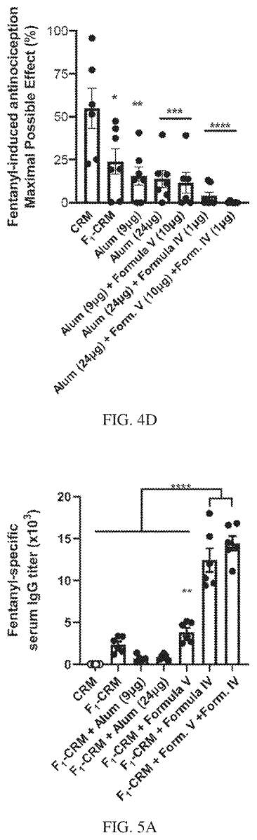

Importantly, when challenged with fentanyl, mice vaccinated with the adjuvanted fentanyl vaccine showed increased protection from the effects of fentanyl compared to those vaccinated with a non-adjuvanted fentanyl vaccine (FIGS. 4A-4D). Interestingly, combining TLR4/7/8 adjuvants with alum, especially TLR7/8 agonist (e.g., the compound of Formula IV), increased overall efficacy with respect to fentanyl challenge.

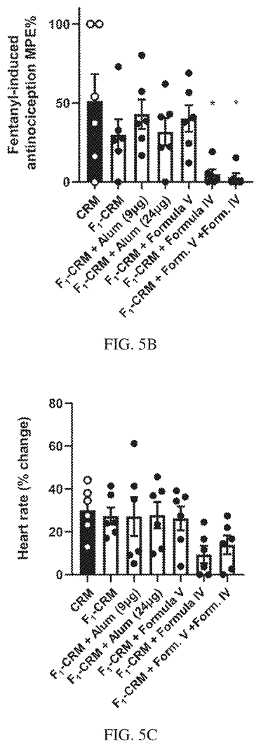

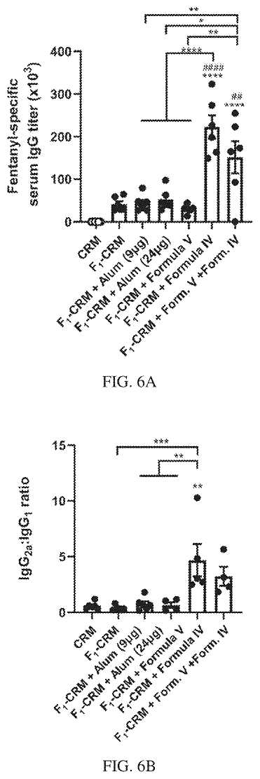

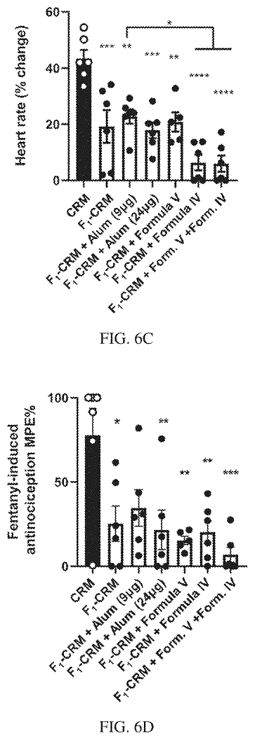

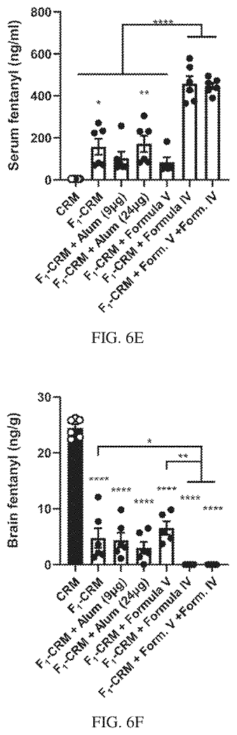

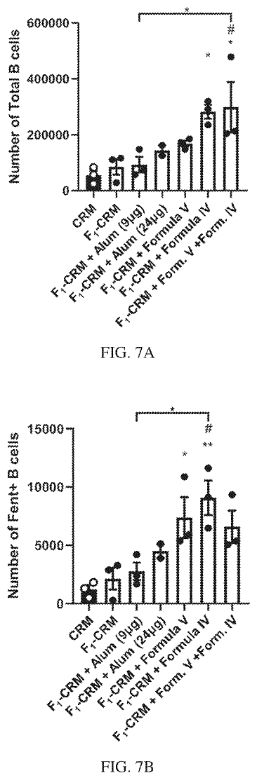

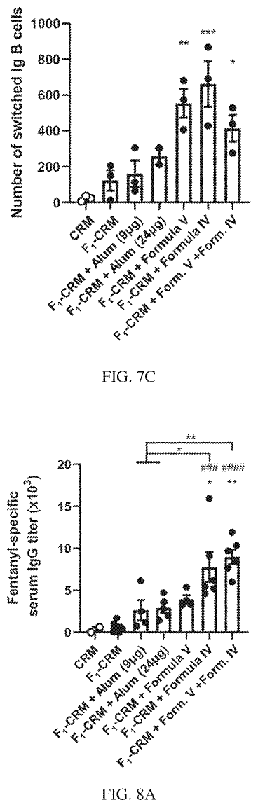

In further challenge experiments, mice were immunized on days 0, 14, and 28, with CRM (negative control), F₁-CRM alone, or F₁-CRM formulated in different adjuvants, including alum (low and high dose), TLR4 agonist (e/g/. the compound of Formula V), TLR7/8 agonist (e.g., the compound of Formula IV), or a combination of both the TLR4 and TLR7/8 agonists. Antibody titers were measured 1 day before each fentanyl challenge occurring on either days 21 (FIGS. 5A-5C) and 35 (FIGS. 6A-6F) to measure vaccine efficacy. After challenge, fentanyl-induced antinociception was measured using a hot plate test of antinociception and heart rate was measured using a pulse oximeter via neck collars. During the challenge on day 35, mice were euthanized at 30 minutes post-drug injection to collect blood and brain for analysis of fentanyl concentrations via LC-MS. Groups that received TLR7/8 agonist (e.g., the compound of Formula IV), either alone or in combination with TLR4 agonist (e.g., the compound of Formula V), displayed increased fentanyl-specific IgG antibody titers, decreased fentanyl-induced antinociceptive effects, and decreased changes in heart rate compared to other groups in both the 21d and 35d challenge. The drug challenge on day 35 also shows that in TLR7/8 agonist (e.g., the compound of Formula IV) groups there is an increased fentanyl concentration in the serum and decreased fentanyl concentration in the brain, indicating the antibodies are successful in inhibiting the drug from crossing the blood-brain barrier (FIGS. 6E-6F). In a separate experimental cohort, immunized mice were euthanized for B cell analysis 7 days after the first vaccination to determine which vaccine formulation would be most effective in inducing expansion of the fentanyl-specific B cell population (FIGS. 7A-7C). Lymph nodes and spleens were processed, magnetically enriched for antigen-specific B cells, and analyzed via flow cytometry. Preliminarily, TLR7/8 agonist (e.g., the compound of Formula IV) appears to increase overall B cell recruitment to the spleen/lymph nodes after vaccination and increases overall number of fentanyl-specific B cells, as well as mature, class switched fentanyl-specific B cells.

### Conclusion

Combinations of adjuvants and antigen may lead to superior efficacy beyond that which could be provided by any of the proposed adjuvants alone, as illustrated by the unexpected efficacy of a vaccine containing multiple TLR4/7/8 agonists. As some previous attempts to develop TLR7/8 adjuvants for human use have been halted due to toxicity and/or reactogenicity, it is important to note that the TLR7/8 agonist (e.g., the compound of Formula IV) was designed specifically to adsorb to aluminum salts with high efficiency and incorporate into liposomes in order to reduce rapid distribution and systemic toxicity previously noted with core TLR7/8 ligands. That TLR7/8 agonist (e.g., the compound of Formula IV) robustly enhanced the production of antibodies against fentanyl while impeding various physiological responses to fentanyl when administered to a mouse model is encouraging for the use of TLR7/8 agonists in human OUD subjects.

### Example 3

### Introduction

After successful immunization in a mouse model, lead adjuvanted vaccine formulations were further tested in rats.

### Methods

Rats were immunized 3 times, 3 weeks apart (days 0, 21, and 42). Blood was collected on day 49 to determine antibody titers, followed by a 0.05 mg/kg fentanyl challenge one week later (day 56). Similar to previous studies in mice, after drug challenge fentanyl-induced antinociception was measured using a hot plate test of analgesia, while heart rate and oxygen saturation were measured using a pulse oximeter via neck collars. Blood and brain samples were collected 30 minutes after drug challenge for analysis of drug concentrations via LC-MS to determine the efficacy of vaccines in altering fentanyl pharmacokinetics.

### Results

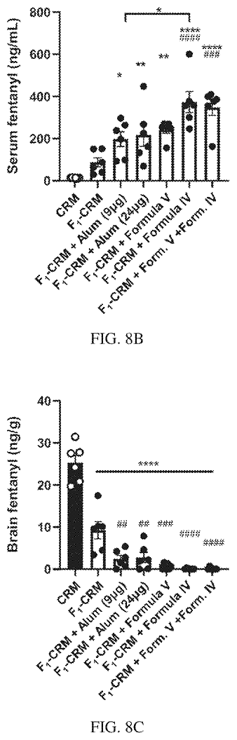

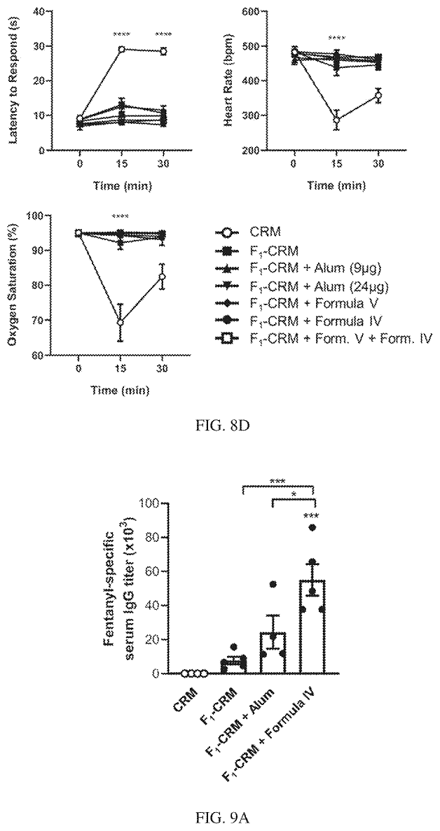

Antibody titers of immunized rats were increased in groups receiving TLR7/8 agonist (e.g., the compound of Formula IV) compared to the control and other adjuvanted groups. After challenging the rats with fentanyl however, all immunized groups performed equally compared to the control group, although those that received TLR7/8 agonist (e.g., the compound of Formula IV) exhibited both the highest levels of serum fentanyl and the lowest levels of brain fentenyl (FIGS. 8A-8D). While these data conclusively indicate the efficacy of TLR7/8 agonist-adjuvanted fentenyl vaccines in a rat model, these results also indicate that this challenge dose was only sufficient to demonstrate modest differences between different immunized groups.

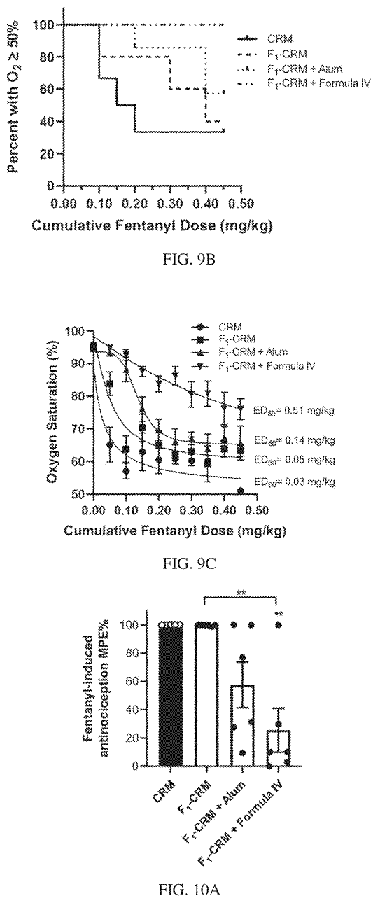

A follow-up rat experiment directly compared the F₁-CRM+alum and F₁-CRM+TLR7/8 agonist formulations. In this study, immunized rats were challenged with a cumulative dosing paradigm involving incremental doses from 0.05 mg/kg to 0.45 mg/kg, where cumulative dosage was increased every 15 minutes (FIGS. 9A-9C). After administering fentanyl, once rats displayed oxygen saturation below 50% (considered to be “death” for practical purposes) they were given naloxone to rescue them from fatal overdose. Significantly more rats “survived” in the TLR7/8 agonist group compared to those in either the control or vaccine with alum adjuvant groups (FIG. 9B). Furthermore, the ED₅₀ of the vaccine was increased from 0.14 mg/kg when adjuvanted with alum to 0.51 mg/kg when adjuvanted with TLR7/8 agonist (FIG. 9C). The shift in ED₅₀ reflected a shift in fentanyl potency in the most effective vaccine formulation.

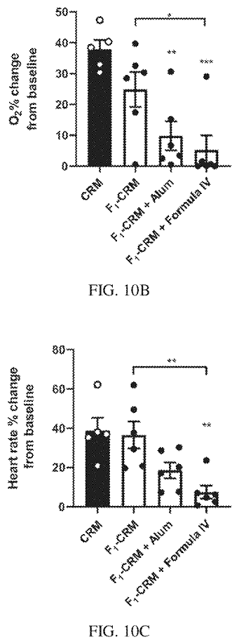

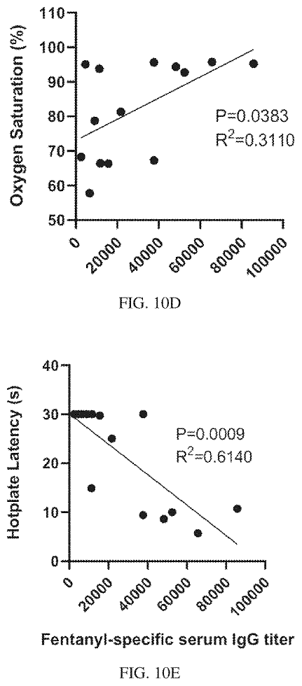

A week after the cumulative dosing challenge, rats were re-challenged with an additional 0.1 mg/kg bolus dose of fentanyl. Fentanyl-induced effects including antinociception, respiratory depression, and bradycardia were significantly reduced in rats immunized with TLR7/8 agonist compared to those in the control group (FIGS. 10A-10E). Additionally, significant correlations emerged between oxygen saturation and hot plate latency when these metrics were plotted against fentanyl-specific IgG titers, indicating that increased titers are correlated with increased vaccine efficacy.

### Conclusion

Just as had been observed in mice, TLR7/8 agonist proved to be a potent adjuvant of fentanyl vaccines when administered to rats, protecting rats from even more acute fentanyl dosages than had been tested previously. These data provide further supporting evidence that vaccination with a TLR7/8 agonist-adjuvanted vaccine, such as F₁-CRM+TLR7/8 agonist, would protect against toxicity and potentially fatal overdose from exposure to fentanyl in human OUD subjects.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating an opioid use disorder (OUD) in a subject in need thereof, the method comprising administering to the subject an effective amount of a composition comprising an opioid antigen and a toll-like receptor 7 and 8 (TLR7/8) agonist.

2. The method of claim 1, wherein the opioid antigen is a fentanyl (FEN) antigen.

3. The method of claim 1 or claim 2, wherein the TLR7/8 agonist is an imidazoquinoline compound.

4. The method of claim 3, wherein the imidazoquioline compound is of Formula II:

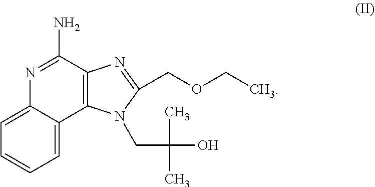 [SMILES: CCOCc1nc2c(N)nc3ccccc3c2n1CC(C)(C)O]

5. The method of claim 1 or claim 2, wherein the TLR7/8 agonist is an oxoadenine compound.

6. The method of claim 5, wherein the oxoadenine compound is of Formula IV:

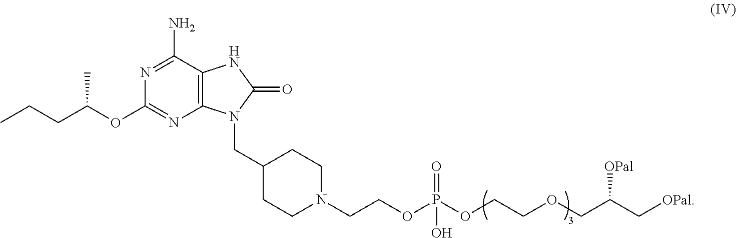

7. The method of any one of claims 1-6, wherein the TLR7/8 agonist is lipidated.

8. The method of any one of claims 1-7, wherein the TLR7/8 agonist is incorporated into a liposome.

9. The method of any one of claims 1-8, further comprising administering to the subject an effective amount of a TLR4 agonist.

10. The method of claim 9, wherein the TLR4 agonist is of Formula V:

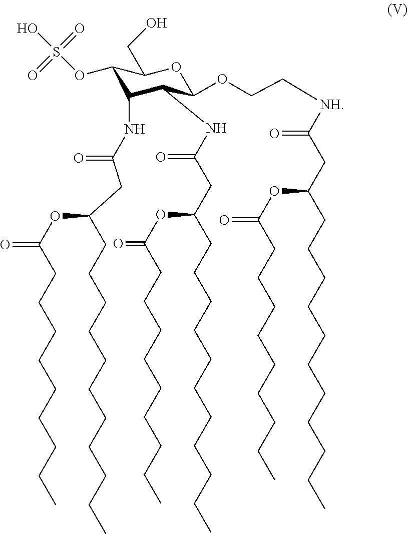

11. The method of any one of claims 1-10, wherein the composition further comprises alum.

12. The method of any one of claims 1-10, wherein the subject is a human adolescent.

13. The method of any one of claims 1-10, wherein the subject is a human adult.

14. The method of any one of claims 1-10, wherein the subject is a human that is between ages 18-30.

15. The method of any one of claims 1-15, wherein the OUD is opioid addition.

16. The method of any one of claims 1-14, wherein the OUD is opioid tolerance.

17. The method of any one of claims 1-14, wherein the OUD is opioid overdose.

18. The method of any one of claims 1-17, wherein the opioid is heroin, 6-acetylmorphine, morphine, oxycodone, hydrocodone, fentanyl, or analogs thereof.

19. The method of any one of claims 2-18, wherein the FEN antigen is a fentanyl-based hapten.

20. The method of any one of claims 2-18, wherein the FEN antigen is a FEN conjugated to a carrier protein, optionally wherein the carrier protein is tetanus toxoid (TT), diphtheria toxoid (CRM), keyhole limpet hemocyanin (KLH).

21. The method of any one of claims 1-20, wherein the administration is intravenous, intramuscular, intradermal, intranasal, topical, or oral.

22. The method of any one of claims 1-21, wherein the subject is administered a second agent for treating the OUD.

23. A composition comprising an opioid antigen and a toll-like receptor 7 and 8 (TLR7/8) agonist, optionally wherein the opioid antigen is fentanyl (FEN) or FEN based hapten.

24. The composition of claim 23, further comprising a TLR4 agonist.

25. The composition of claim 23 or claim 24, further comprising alum.

26. The composition of any one of claims 23-25, wherein the composition is a vaccine.

27. A toll-like receptor 7 and 8 (TLR7/8) agonist for use as an adjuvant in an opioid vaccine.

28. A toll-like receptor 7 and 8 (TLR7/8) agonist for use in enhancing an immune response in a subject having an opioid use disorder (OUD).

29. A method of treating an opioid use disorder (OUD) in a subject in need thereof, the method comprising administering to the subject an effective amount of a composition comprising a toll-like receptor 7 and 8 (TLR7/8) agonist.
